(19) United States
(12) Patent Application Publication
(10) Pub. No.: US 2023/0118462 A1
(43) Pub. Date: Apr. 20, 2023

(54) Process for Making Sorbic Acid from Renewable Materials

(71) Applicant: Celanese International Corporation, Irving, TX (US)

(72) Inventors: Aujke van Kooij, Haarlem (NL); Sylvia Nefkens, Amsterdam (NL); Brigitte Beringer-Molenaar, Badhoevedorp (NL)

(21) Appl. No.: 17/959,347

(22) Filed: Oct. 4, 2022

Related U.S. Application Data
(60) Provisional application No. 63/252,300, filed on Oct. 5, 2021.

(51) Int. Cl. C07C 51/09

(52) U.S. Cl. CPC C07C 51/09

(57) ABSTRACT

A process for making sorbic acid from renewable materials is provided. The process comprises converting acetic acid to ketene; converting acetaldehyde to crotonaldehyde; reacting the ketene with the crotonaldehyde to produce a polyester; and converting the polyester to sorbic acid. Renewable materials are incorporated by one of the following methods: a) the acetic acid is produced by reacting methanol derived from renewable organic material with carbon monoxide, b) the acetic acid is a biobased acetic acid, c) the crotonaldehyde is a biobased crotonaldehyde, d) the crotonaldehyde is produced by converting a biobased acetaldehyde to crotonaldehyde, e) the crotonaldehyde is produced by converting acetaldehyde to crotonaldehyde and the acetaldehyde is produced from bioethylene, or any combination of a), b), c), d) and e).

### RELATED APPLICATION

The present application is based upon and claims priority to U.S. Provisional Patent Application Ser. No. 63/253,300, having a filing date of Oct. 5, 2021, which is incorporated herein by reference.

### BACKGROUND OF THE INVENTION

Sorbic acid and salts thereof, such as potassium sorbate, are widely used as preservatives in food, feed, pharmaceutical, personal care, household cleaning, and leather care products, as well as coatings, inks, adhesives, silicone emulsions, surfactants, and enzyme preparations. They have a long history of safe use in these applications. Sorbic acid and potassium sorbate have conventionally been manufactured from petrochemical resources. For example, sorbic acid is typically produced by the reaction of ketene and crotonaldehyde. Sorbic acid has been isolated from natural resources, such as the mountain ash berry; however, sourcing from these berries on an industrial scale is not practical.

Recently, however, there have been significant efforts of companies to develop carbon neutral or carbon negative processes and consumers have favored products made from renewable ingredients. As such, it would be desirable to produce sorbic acid and sorbates in a more sustainable way. In this regard, a need exists for a process for producing sorbic acid and salts thereof from sources other than fossil-based sources.

### SUMMARY OF THE INVENTION

In accordance with one embodiment of the present invention, a process for making sorbic acid from renewable materials is provided. The process comprises converting acetic acid to ketene, reacting the ketene with crotonaldehyde to produce a polyester, and converting the polyester to sorbic acid. The renewable material is provided by producing the acetic acid by reacting methanol derived from organic waste with carbon monoxide, using a biobased acetic acid, using a biobased crotonaldehyde, using a crotonaldehyde produced by converting a biobased acetaldehyde to crotonaldehyde, and/or using a crotonaldehyde produced by converting an acetaldehyde produced from bioethylene to crotonaldehyde.

In another embodiment, a product containing a preservative comprising sorbic acid or an alkali metal salt of sorbic acid is provided. About 15% or more of the carbon content in the sorbic acid or an alkali metal salt of sorbic acid is biobased, as determined according to ASTM D6866-21.

Other features and aspects of the present invention are set forth in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 shows a process flow diagram of one embodiment of the process according to the present disclosure;

FIG. 2 shows a process flow diagram of another embodiment of the process according to the present disclosure;

FIG. 3 shows a process flow diagram of another embodiment of the process according to the present disclosure;

FIG. 4 shows a process flow diagram of another embodiment of the process according to the present disclosure; and

FIG. 5 shows a process flow diagram of another embodiment of the process according to the present disclosure.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present invention.

Generally speaking, the present disclosure is directed to a process for making sorbic acid from renewable materials. More particularly, a process is described wherein either a portion or all of the raw materials incorporated into the sorbic acid are biobased.

While both ketene and crotonaldehyde can be produced from petrochemical materials through known processes, the present inventors found that biobased raw materials can be used to form the ketene and acetaldehyde and that the sorbic acid can thus be derived from renewable, biobased materials.

As used herein, renewable materials refer to materials that are not derived from fossil materials or petrochemicals. For example, renewable materials may be formed from plant materials or other non-fossil-based organic materials, such as food waste. Biobased content refers to the percentage of carbon atoms in the resulting sorbic acid product (sorbic acid or salt thereof) that originated from renewable materials.

Through the processes described herein, sorbic acid can be produced having various levels of biobased carbon content. For example, the biobased carbon content can be greater than about 15%, in some embodiments greater than about 30%, in some embodiments greater than about 60%, and in some embodiments, greater than about 80% and up to 100%. In some embodiments, all of the carbon content in the resulting the resulting sorbic acid is derived from biobased materials. The percentage of biobased carbon content can be determined either by using a mass balance based on carbon tracing or by physical analysis carried out according to ASTM D6866-21 (technically equivalent to ISO 16620-2), which can discriminate between product carbon resulting from contemporary carbon input (renewably sourced) and that derived from fossil-based input by measuring the product's ¹⁴C/¹²C or ¹⁴C/¹³C content. Counts from ¹⁴C in a sample can be compared directly or through secondary standards to SRM 4990C. A measurement of 0% ¹⁴C relative to the appropriate standard indicates carbon originating entirely from fossils (e.g., petroleum based). A measurement of 100% ¹⁴C relevant to the appropriate standard indicates carbon originating entirely from modern sources.

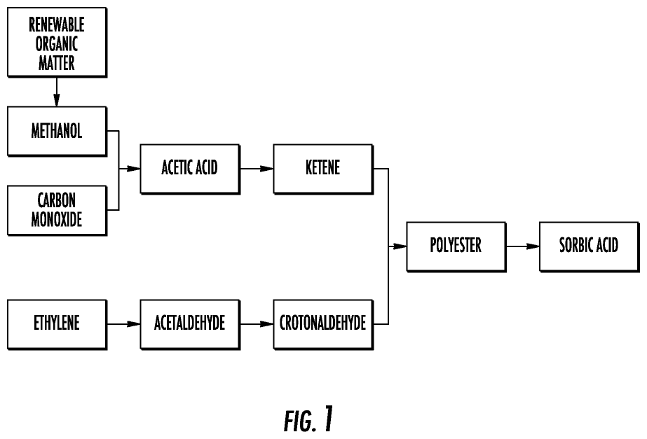

One embodiment of the process is illustrated by the process flow diagram shown in FIG. 1. As shown, acetic acid is formed from the reaction of methanol and carbon monoxide. The acetic acid is then converted to ketene. The ketene is then reacted with crotonaldehyde as known in the art to produce a polyester which is converted to sorbic acid. The crotonaldehyde is produced by converting ethylene to acetaldehyde and then converting the acetaldehyde to crotonaldehyde. In this embodiment, renewable carbon content is introduced by forming the methanol from renewable materials. For example, as shown in FIG. 1, the methanol can be produced from renewable organic matter, such as biomass or organic waste from municipal or industrial sources. By using renewably sourced methanol, at least 16.7% of the carbon content in the resulting sorbic acid is biobased, as determined by a mass balance based on carbon tracing or by physical analysis carried out according to ASTM D6866-21 (technically equivalent to ISO 16620-2). Each step of the process will be described in more detail below.

The methanol can be produced from renewable matter in any manner known in the art. For example, one method to produce renewable methanol is through gasification of renewable organic matter to produce a syngas containing hydrogen (H₂) and carbon monoxide (CO) and then reacting the hydrogen with the carbon monoxide in the presence of a catalyst to form methanol. The organic source can be any suitable renewable source, such as biomass, municipal solid waste, or industrial waste. The source can be converted to syngas through known gasification methods.

In one exemplary embodiment, during gasification, a carbonaceous material undergoes pyrolysis, during which the organic material is heated to release volatiles and produce char. Combustion then occurs during which the volatiles and char react with oxygen to form carbon dioxide (CO₂) according to the reaction: C+O₂→CO₂. Pyrolysis typically involves heating to temperatures above about 700° C., such as from about 800° C. to about 1600° C.

Next, char reacts with carbon dioxide and steam (H₂O) to produce carbon monoxide and hydrogen gas via the reactions: C+H₂O→H₂+CO and C+CO₂→2CO.

In essence, the biomass gasification process employs oxygen or air to combust some of the biomass and produce carbon monoxide and energy, the latter of which is utilized to convert the remaining biomass to hydrogen and additional carbon monoxide. Catalysts can optionally be used to facilitate the process.

Many different gasifiers can be used for gasification, including bubbling fluidized bed gasifiers, circulating fluidized bed gasifiers, fixed bed gasifiers, and entrained flow gasifiers. Methods for producing a syngas via gasification of renewable organic matter are provided in, for example, U.S. Pat. Nos. 4,452,611; 4,699,632; 7,736,402; 8,241,599; 8,845,772; 10,093,875 and U.S. Patent Publication Nos. 2005/0095183 and 2007/0270511; which are entirely incorporated herein by reference.

Syngas can also be produced from renewable organic materials through bioprocesses, such as fermentation. For example, organic waste can be fermented by microorganisms that produce methane and carbon dioxide. The gaseous products of such fermentation process are called biogas. The biogas can then be optionally cleaned and then converted to syngas through a steam reformation process.

In the steam reformation process, the biogas is heated and pressurized, mixed with steam, and passed through a reformer to convert the methane and steam to a syngas according to the following reaction:

CH₄+H₂O→CO+3H₂.

Methanol can then be produced from the syngas according to any method known in the art. The conversion can be done in the gas or liquid phase. The reactions involved are:

2H₂+CO→CH₃OH;

CO₂+3H₂→CH₃OH+H₂O; and

CO+H₂O→CO2+H₂

Typically, the conversion is carried out in fixed-bed reactors at high pressure. For example, the reaction is generally carried out at pressures from about 25 to about 120 bar and temperatures from about 200 to 400° C.

The reaction is typically carried out in the presence of a catalyst. Many catalyst systems are based on copper, zinc oxide, alumina and/or magnesia. Recent catalysts have also been developed that include carbon, nitrogen, and platinum. Exemplary catalyst systems can include: Cu—Zn—Al—Cr, Cu—Zn—Al, Cu—Zn—Al—Cr—Mn, Cu—Zn—Cr, Cu—Zn—V, Cu—Mn—V, Cu—Zn-Mp, Cu—Zn—B, Cu—Zn—Ag, Cu—Zn—Re, Cu—Zn—Zr, Cu—Zn—Al—Ga, Cu—Zn—Al—Zr—Mo, Pd/CeO₂, Cu—Zn—Al—Zr, or any other catalyst system known to produce methanol from syngas.

Another source of renewable methanol is as a byproduct of the kraft pulping process. For example, when a hydroxyl ion reacts with a lignin methoxyl group, methanol is produced by the following reaction:

lignin.OCH₃+OH⁻→CH₃OH+lignin.O⁻.

In pulp mills, the resulting methanol typically ends up in a stripper off-gas stream which contains methanol, non-condensable gases, and water vapor. Methanol can then be separated out and purified through known methods, such as by distillation. The purified methanol can then be used in the process described herein.

As shown in FIG. 1, the methanol can then be reacted with carbon monoxide to form acetic acid. This reaction can be carried out in any manner known in the art. Typically, for example, the carbon monoxide and methanol are reacted in the presence of a catalyst and a promoter, and the methanol is carbonylated according to the following equation:

CH₃OH+CO→CH₃COOH.

In one embodiment, for example, the starting materials in the reaction mixture are liquid methanol and gaseous carbon monoxide, which are both fed to a reactor.

Suitable catalysts include rhodium catalysts and iridium catalysts. A non-limiting example of the rhodium catalyst is a rhodium complex represented by the chemical formula [Rh(CO)₂|₂]⁻. A non-limiting example of the iridium catalyst is an iridium complex represented by the chemical formula [Ir(CO)₂|₂]⁻. The reaction mixture typically has a catalyst concentration from about 200 to about 5000 ppm of the entire liquid phase in the reaction mixture.

The promoter is generally an iodide, which assists the activity of the catalyst. Non-limiting examples of the iodide include methyl iodide and an ionic iodide. Methyl iodide may accentuate the action of the catalyst. The reaction mixture may have a methyl iodide concentration of from about 1 to about 20 wt. % percent of the entire liquid phase in the reaction mixture. An ionic iodide is an iodide that forms an iodine ion in the reaction liquid. Ionic iodides may stabilize the catalyst and restrict side reactions. Non-limiting examples of ionic iodides include lithium iodide, sodium iodide, and potassium iodide. The reaction mixture may have an ionic iodide concentration of from about 1 to about 25 wt. % percent of the entire liquid phase in the reaction mixture.

Water is typically provided for dissolving water-soluble components in the reaction system. For example, the reaction mixture may have a water concentration of from about 0.1 to about 15 wt. % percent of the entire liquid phase in the reaction mixture. The water concentration is preferably about 15 wt. % or less in order to reduce the energy involved in separating the resulting reaction contents.

The reaction temperature is typically from about 150° C. to about 250° C.; the total reaction pressure is typically from about 2.0 to about 3.5 MPa; and the carbon monoxide partial pressure is typically from about 0.5 to about 1.8 MPa, such as from about 0.8 to about 1.5 MPa.

The acetic acid produced can be purified by any method known in the art prior to its conversion to ketene.

As shown in FIG. 1, the acetic acid is then converted to ketene. This process can be carried out by any method known in the art. In one exemplary embodiment, acetic acid is converted to ketene by pyrolysis of the acetic acid. The pyrolysis of the acetic acid is typically carried out in the vapor phase at atmospheric, subatmospheric or superatmospheric pressure, at temperatures between about 600 and about 800° C. in the presence of a suitable pyrolysis catalyst. Suitable catalysts include phosphates such as diammonium phosphate, triethyl phosphate, tricresyl phosphate, or other esters of phosphoric acid. The amount of catalyst used is generally from about 0.1 to about 1.0%, such as from about 0.2 to about 0.5%, by weight based on the weight of the feed stock. In one embodiment, the vapors of the feed stock, e.g., acetic acid, and the catalyst are passed at a pressure between about 1 and about 2.5 bar through a reactor maintained at a temperature from about 625 to about 800° C. The reaction can proceed for about 0.01 to about 5.0 seconds and is then treated with a neutralizing agent such as ammonia, pyridine, aniline, or a suitable aliphatic amine which neutralizes the catalyst and retards recombination of the ketene with unreacted acetic acid and water. Preferably, an excess of the neutralizing agent is employed.

The resulting ketene can then be reacted with the crotonaldehyde to form a polyester precursor to the sorbic acid. As shown in FIG. 1, the crotonaldehyde is produced from conversion of ethylene to acetaldehyde and then to crotonaldehyde. In the embodiment shown in FIG. 1, the ethylene can be petrochemically or renewably sourced. Ethylene can be converted to acetaldehyde by any method known in the art. For example, acetaldehyde is generally industrially produced by the Wacker oxidation of ethylene, which uses an aqueous catalyst system of palladium chloride, copper chloride, and hydrochloric acid to accomplish the following net conversion:

C₂H₄+½O₂→CH₃CHO. (1)

Aspects of the Wacker process are disclosed in U.S. Pat. Nos. 3,122,586; 3,119,875; and 3,154,586, each incorporated by reference entirely.

In the Wacker process, ethylene is oxidized by cupric chloride in aqueous solution, catalyzed by palladium, as indicated in reaction (2):

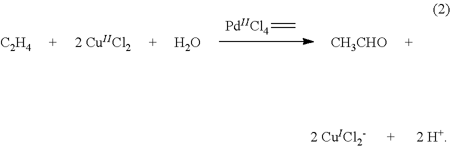

In a typical process, copper is present in the aqueous solution at concentrations of about 1 mole per liter, total chloride is present at concentrations of about 2 moles per liter, and the palladium catalyst is present at concentrations of about 0.01 moles per liter. Under these conditions, palladium(II) exists predominantly as the tetrachloropalladate ion, PdCl₄⁼. Cuprous chloride resulting from the oxidation of ethylene is solubilized in the aqueous solution by the co-produced hydrochloric acid, as the dichlorocuprate ion, CuCl₂. In a subsequent Wacker chemistry step, this reduced copper is reoxidized by reaction with oxygen (O₂) as shown in reaction (3):

2Cu^{|}Cl₂⁻+2H⁺+½O₂→2Cu^{∥}Cl₂+H₂O (3)

Reactions (2) and (3) combine to produce overall reaction (1). Two acetaldehyde manufacturing processes, a two-stage process and a one-stage process, have been developed and operated using the Wacker system chemistry. In the two-stage process, ethylene oxidation by cupric chloride, reaction (2), and reoxidation of cuprous chloride by air, reaction (3), are conducted separately, with intermediate removal of the acetaldehyde product from the aqueous solution. The reoxidized aqueous solution is recycled to the ethylene oxidation stage. The reactions are conducted at temperatures from about 100 to about 130° C. in reactors which, by providing very efficient gas-liquid mixing, result in high rates of diffusion (mass transfer) of the reacting gas into the aqueous solution.

In the one-stage process, ethylene and oxygen are simultaneously reacted with the aqueous solution, from which acetaldehyde is continuously removed. Palladium catalyzes the oxidation of ethylene by cupric chloride (reaction (2)) by oxidizing ethylene (reaction (4)) and then reducing cupric chloride (reaction (5)):

C₂H₄+PdCl₄⁼+H₂O→CH₃CHO+Pd⁰+2H⁺+4Cl⁻ (4)

Pd⁰+4Cl⁻2Cu^{∥}Cl₂→PdCl₄⁼+2Cu^{|}Cl₂⁻ (5)

Functionally, the copper chlorides mediate the indirect reoxidation of the reduced palladium(0) by oxygen via reaction (5) plus reaction (3). Direct oxidation of palladium(0) by oxygen is thermodynamically possible but is far too slow for practical application.

The resulting acetaldehyde is optionally purified and then converted to crotonaldehyde, as shown in FIG. 1. The conversion of acetaldehyde to crotonaldehyde can be carried out through any method known in the art. For example, crotonaldehyde is conventionally produced through aldol condensation of acetaldehyde using a basic catalyst to form 2-hydroxybutyraldehyde, which is then heated and dehydrated in an acidic solution to produce crotonaldehyde. This process can be expressed by the following equations:

CH₃CHO→CH₃CH(OH)CH₂CHO

CH₃CH(OH)CH₂CHO→CH₃CH═CHCHO+H₂O

Suitable basic catalysts for facilitating the aldol condensation reaction include sodium hydroxide; metal oxides; anion exchange resins; organic amines such as trimethylamine and triethylamine; quaternary ammonium bases such as tetramethylammonium hydroxide; solid-supported alkaline ionic liquids such as 1-butyl-3-methylimidazole hydroxide; silica-supported alkali metal oxides; molecular sieves, such as HX, NaX, and KX; hydrotalcite; and alkaline earth metal oxides supported on molecular sieves or alumina carriers.

Two stage processes are known in which the aldol condensation reaction is carried out in the presence of a basic catalyst followed by a second stage of dehydrating the formed 2-hydroxybutyraldehyde using an acid, such as acetic acid. Singe stage processes are also known in which a solid catalyst, such as a supported metal oxide, can function as both an acid and a base to facilitate the conversion in a single process step.

Exemplary processes are described in U.S. Pat. Nos. 1,693,907; 2,341,229; and 2,810,760; British Patent Publication Nos. GB270764A and GB660972A; and Chinese Patent Publications CN106946675A, CN101462044B, CN100344598C, CN106631739B, CN106883112B, CN1314646C, and CN105037119A; all of which are entirely incorporated by reference.

As shown in FIG. 1, the ketene is then reacted with the crotonaldehyde to form a polyester precursor to sorbic acid. Any suitable method known in the art for preparing a poly sorbic acid ester from crotonaldehyde and ketene can be used. For example, a polyester can be prepared by reacting crotonaldehyde with ketene in the presence of a fatty acid salt of a divalent and/or trivalent metal of subgroup II to VIII of the Periodic Table as a catalyst in the presence of an inert solvent. The catalysts are generally present in amounts from about 0.1 to about 5 wt. %, such as from about 0.5 to about 2 wt. %, based on the amount of crotonaldehyde used.

Exemplary catalysts include fatty acid salts of zinc, cadmium, mercury, cobalt, nickel and iron. The fatty acid typically has from about 4 to about 18 carbon atoms. When the sorbic acid is intended to be used in a food or consumer product, it is preferable to avoid toxic metals. In one embodiment, the catalyst is zinc isobutyrate or zinc isovalerate. Other salts are, for example, those of α-methylbutyric acid, diethylacetic acid, 2-itthylcaproic acid, stearic acid, oleic acid, and other fatty acids with 4 to 18 carbon atoms.

Aromatic, aliphatic, and alicyclic hydrocarbons or their derivatives and excess crotonaldehyde are suitable solvents for the reaction, such as hexane, heptane, octane, benzene, toluene, xylene, cyclohexane, methylene chloride, chloroform, carbon tetrachloride, chlorobenzene, and nitrobenzene.

Generally, it is preferable to use equimolar amounts of ketene and crotonaldehyde, in which the catalyst is dissolved. Generally, the reaction is carried out at a temperature from about 25 to about 350° C.

As shown in FIG. 1, the resulting poly sorbic acid ester is then converted to sorbic acid. This can be done by any method known in the art. For example, in one exemplary process, thermal catalytic cleavage of the polyester is carried out, which comprises cleaving the polyester in the presence of an inert solvent and catalyst. The solvent is generally used in an amount of about 1 to about 15 times the amount by weight of the polyester and the catalyst is generally used in an amount from about 20 to 60 wt. % based on the polyester. Suitable solvents typically have a boiling point of about 150° C. or higher, such as about 180° C. or higher. Suitable catalysts include secondary or tertiary aliphatic, alicyclic, 5- or 6-membered heterocyclic nitrogen- and/or oxygen-containing or aliphatically aromatically substituted amines with boiling points of about 100° C. or higher, such as about 150° C. or higher. The reaction is carried out at temperatures of from about 160° C. to about 220° C. while simultaneously distilling off the sorbic acid and the solvent.

In one embodiment, the cleavage is carried out in a continuous distillation apparatus. The sorbic acid polyester dissolved in the solvent is charged into the distillation vessel where the amine-catalyzed cleavage of the sorbic acid polyester to give sorbic acid takes place. The sorbic acid formed is distilled off with the solvent via a rectification column operating from about 160 to about 200° C. and from about 20 to about 50 hPa with reflux. Rectification prevents the transfer of amine into the distillate and helps achieve the appropriate purity.

The sorbic acid is then crystallized out of the distillate and separated off from the solvent. The solvent can be recirculated.

Suitable amines which may be mentioned by way of example are: methyloctadecylamine, dimethyloctadecylamine, dimethylhexadecylamine, dimethyltetradecylamine, dimethyldodecylamine, dibutyidodecylamine, N,N′,N,N′-tetramethylhexamethylenediamine, N,N,N′-trimethyl-N′-phenylethylenediamine, N-octadecylpyrrolidone, N-octadecylpiperidine, N-dodecylmorpholine, N,N′-dipropylpiperazine, α-hexylpyrrolidone, triethylenetetramine, ethylbis(β-ethylaminoethyl)amine, 1-octyldiethylenetriamine, ethylene glycol bis(2-methylaminoethyl ether), dioctadecylamine, diethylenetriamine, trioctadecylamine, trioctylamine, tricyclohexylamine.

Suitable solvents to carry out the cleavage of the sorbic acid polyester are aliphatic, alicyclic, aromatic hydrocarbons, their chlorine, bromine and nitro derivatives, and also ethers and silicone oils whose boiling point at atmospheric pressure is about 150° C. or higher, such as about 180° C. or higher. However, ketones, esters, carboxylic acids and alcohols having the appropriate boiling range can also be used as solvents. It is preferable to use solvents which are liquid at ambient temperatures, boil below about 300° C. and form azeotropic mixtures with sorbic acid, so that they at the same time act as entrainers. Such solvents include petroleum fractions, dodecane, tetradecane, 5-methyldodecane, dodecene, dicyclohexylmethane, p-di-tert-butylbenzene, 1-methyinaphthalene, 2-methylnaphthalene, 1-ethylnaphthalene, tetrahydro-naphthalene, diphenylnaphthalene; halogenated aliphatic, cycloaliphatic or aromatic hydrocarbons such as dichlorododecane, 1,5-dibromopentane, benzotrichloride, o- and m-dibromobenzene; nitro compounds such as nitrobenzene, 2-nitrotoluene; nitrites such as benzyl cyanide; carbonyl compounds such as acetophenone or the heterocyclic 2-acetylthiophene; heterocyclic compounds such as chromane, thiophene; ethers such as resorcinol dimethyl ether, diphenyl ether, safrole, isosafrole; acids such as enanthric acid, α-ethylcaproic acid, caprylic acid, capric acid; or esters such as ethyl benzoate, methyl phenylacetate and methyl salicylate.

The cleavage of the polyester can also be carried out by hydrolysis with a mineral acid, such as hydrochloric acid. For example, the polyester can be hydrolyzed at temperatures ranging from about 10° C. to about 110° C. with hydrochloric acid. The concentration of hydrochloric acid can be from about 15 to about 40% by weight and the amount of hydrochloric acid in terms of hydrogen chloride can be from about 10 to about 160 parts by weight relative to 100 parts by weight of the polyester.

Alternatively, the polyester can be converted to sorbic acid by saponification using a strong base and then heating the saponified product in the presence of an acid to form sorbic acid. For example, saponification can be carried out by heating for a suitable time to a temperature of about 90 to about 1000° C. with about 10% to about 40% sodium hydroxide solution. An acid can then be added such that the free oxyacids are largely precipitated. The mixture can then be heated for a suitable time period, such as about an hour, to temperatures form about 90 to about 1000° C. in a strong acid to produce sorbic acid.

Strong bases such as caustic soda, potassium hydroxide and barite are particularly suitable for saponifying the polymer formed in the first process stage.

Hydrochloric acid, sulfuric acid, benzenesulfonic acid and p-toluenesulfonic acid, for example, can be used for the acid treatment of the solution obtained with the alkalis mentioned.

The sorbic acid can be purified by any method known in the art.

Exemplary processes for producing sorbic acid from ketene and crotonaldehyde are provided in U.S. Pat. Nos. 3,461,158; 6,673,963; and 6,794,540; U.S. Patent Publication No. 2003/0065218; and German Patent Nos. 1042573 and 1059899; which are entirely incorporated herein by reference.

While in one embodiment of the process shown in FIG. 1, the methanol is renewably sourced and the remaining raw materials, such as carbon monoxide and ethylene are formed from fossil resources, in other embodiments, the ethylene and carbon monoxide can also be sourced from renewable materials, allowing for higher biobased carbon contents.

In another embodiment, illustrated by FIG. 2, the acetic acid is a biobased acetic acid derived from renewable organic matter, thus removing the steps of forming methanol and reacting it with carbon monoxide. Directly producing the acetic acid from renewable organic matter allows for a higher renewable carbon content in the resulting sorbic acid than the process shown in FIG. 1 because it eliminates the use of any petrochemically sourced carbon monoxide. For example, by using a renewably sourced acetic acid, the biobased carbon content in the resulting sorbic acid is at least 33.3%, as determined by a mass balance using carbon counting or by physical analysis carried out according to ASTM D6866-21 (technically equivalent to ISO 16620-2).

Various methods are known for producing bio-based acetic acid, any of which can be used in the process described herein. For example, acetic acid can be produced by fermentation, either directly by an acetogen after pretreatment and hydrolysis of the organic material or by first producing ethanol and then fermenting the ethanol to acetic acid. Alternatively, ethanol or 2,3-butanediol produced by fermentation of organic matter can be oxidized to acetic acid. Acetic acid can also be recovered from wood extract. The acetic acid can then be purified by known methods.

In one embodiment, ethanol is produced by fermentation, purified, and then oxidized to acetic acid. Oxidation of ethanol to acetic acid can be carried out by reacting liquid ethanol with oxygen or air in the presence of a catalyst. The catalyst may be a noble metal oxidation catalyst, such as Pt, Pd, Rh, or Ir, on a hydrophobic support, such as styrene-divinylbenzene co-polymer, fluorinated carbon, or silicalite, or on activated carbon. In another embodiment, the catalyst is a palladium catalyst placed on a titanium-, phosphorus-, and oxygen-based support.

Oxidation can also be performed in the gas phase by contacting ethanol vapor with an oxygen source in the presence of a catalyst. Such a process can be carried out in, for example, a fixed bed reactor or a fluidized bed reactor. In some embodiments, the catalyst is a palladium-based catalyst. For example, in one embodiment, the catalyst is a supported vanadium-, titanium-, and oxygen-based catalyst. In another embodiment, a promoter, such as Se, Te, Sb, Cr, Au, Mn, and/or Zn, is incorporated into the palladium-based catalyst. Other suitable catalysts for gas phase oxidation are calcined oxide compositions of the formula MoₐV_{b}Nb_{c}Sb_{d}Xₑ wherein X is at least one of the following metals Li, Na, Be, Mg, Ca, Sr, Ba, Zn, Cd, Hg, Sc, Y, La, Ce, Al, Tl, Ti, Zr, Hf, Pb, Ta, As, Bi, Cr, W, U, Te, Fe, Co and Ni; a is equal to 0.5 to 0.9, b is equal to 0.1 to 0.4, c is equal to 0.001 to 0.2, d is equal to 0.001 to 0.1 and e is equal to 0.001 to 1.0. Suitable support materials include silica, alpha-alumina, gamma-alumina, silica-alumina, monoclinic zirconia, tetragonal zirconia, diatomaceous earth, montmorillonite and titania, or ion exchange resins, polymer-based carriers, and the like. However, it is not always necessary to use a support.

In another embodiment, ethanol is converted to acetaldehyde which can then be oxidized to acetic acid. For example, in one embodiment, a dehydrogenation reaction is performed with ethanol in a gaseous state on fixed bed of a copper and silicon-based catalyst, at a temperature of from about 100° C. to about 300° C. Alternatively, the ethanol can be dehydrogenated by passing it through a fixed bed of a silver-based catalyst at an elevated temperature, such as about 500° C. The resulting acetaldehyde can then be transformed into acetic acid by oxidation under air, in a liquid state, at a temperature of about 60° C. and under a pressure of about 2.5 bar. The catalyst used in this second stage can be cobalt and/or manganese based. However, rather than performing the second reaction to convert acetaldehyde to acetic acid, the acetaldehyde can alternatively be used directly for conversion to crotonaldehyde.

In another embodiment, acetic acid is produced from renewable materials by converting 2,3-butanediol and/or acetoin to acetic acid by chemical oxidation. The 2,3-butanediol and acetoin can be produced from renewable organic matter by known fermentation processes. For example, any carbohydrate-containing raw material can serve as the fermentation reactant, such as carbohydrate-containing fractions from the destructurization of lignocellulosics.

The carbohydrate-containing raw materials are initially converted in one of the known fermentation processes for the production of 2,3-butanediol into a fermentation mixture containing compounds having 2 to 5 carbon atoms, such as stereoisomers of 2,3-butanediol (S,S; R,R; or meso) or acetoin (3-hydroxy-2-butanone, R- or S-form).

The oxidation of 2,3-butanediol and/or acetoin can be carried out in a reactor which is suitable for performing oxidation reactions, such as a stirred tank reactor, a bubble column reactor, or a tubular/tube-bundle reactor.

Any catalyst described for the partial oxidation of hydrocarbons is suitable, such as vanadium-, molybdenum-, antimony-, niobium-, titanium- and/or precious metal-containing catalysts.

In another embodiment, acetic acid can be recovered from wood extract. For example, when wood chips or lignocellulosic biomass are pretreated with high temperature steam/water or aqueous solutions to release sugars for fermentation purposes, compounds such as acetic acid/acetate and other organic acids or their conjugate base are also released. Typically, the wood extract contains acetic acid, dissolved monomeric or oligomeric hemicelluloses, and cellulose. The dissolved hemicelluloses contain uronic acids. The wood extract can also contain furfural and other carbohydrate degradation products, methanol derived from wood derived pectins, and methylglucoronic acids.

The acetic acid can be recovered by contacting the wood extract with a water immiscible solvent and an extractant. A solvent/extractant/acetic acid phase forms which can then be separated from the remaining wood extract. The acetic acid can then be separated from the solvent and extractant by any suitable method, such as by distillation.

Any suitable solvent can be used. For example, the solvent may be selected from the paraffin hydrocarbons, including straight-chain paraffin compounds such as undecane, hexane, heptane, octane, nonane, decane, dodecane, tridecane, tetradecane and pentadecane, branched paraffin compounds such as isooctane, isohexane and isododecane and cyclic paraffin compounds such as cyclohexane. Alternatively, the solvent may be selected from the olefin hydrocarbons, including straight-chain olefin compounds such as hexene, octene, decene, dodecene and tetradecene, branched olefin compounds such as diisobutylene and triisobutylene and cyclic olefin compounds such as cyclohexene and dicyclopentene. Further examples of solvents that may be suitable include aromatic hydrocarbons such as toluene, xylene and cumene, and various terpene type compounds.

The extractant for the acetic acid can be dispersed or dissolved in the water insoluble solvent. Any suitable extractant for acetic acid can be used. For example, solvents with a high distribution coefficient can be used to extract the acetic acid. These include trioctylphosphine oxide (TOPO) and long-chain aliphatic amines (including secondary, tertiary and quaternary amines).

In any of the above methods, the resulting acetic acid can be dewatered and purified using customary methods, such as liquid-liquid extraction, extractive rectification, rectification, azeotropic rectification, crystallization and membrane separation.

Various processes to produce biobased acetic acid are described, for example, in U.S. Pat. Nos. 5,770,761; 5,840,971; 8,663,955, and 8,785,688; Canadian Patent No. 1,305,180; Brazilian Patent Publication No. BRP18901776; and PCT Publication Nos. WO 00/61535 and WO 2013/053032; all of which are entirely incorporated herein by reference.

As shown in FIG. 2, the acetic acid is converted to ketene and reacted with crotonaldehyde. The conversion of the acetic acid to ketene, the production of the crotonaldehyde, and the production of sorbic acid from the crotonaldehyde and the ketene are carried out as described above with respect to FIG. 1. While in some embodiments, the ethylene used in the process shown in FIG. 2 is formed from fossil resources, in other embodiments, it can be biobased.

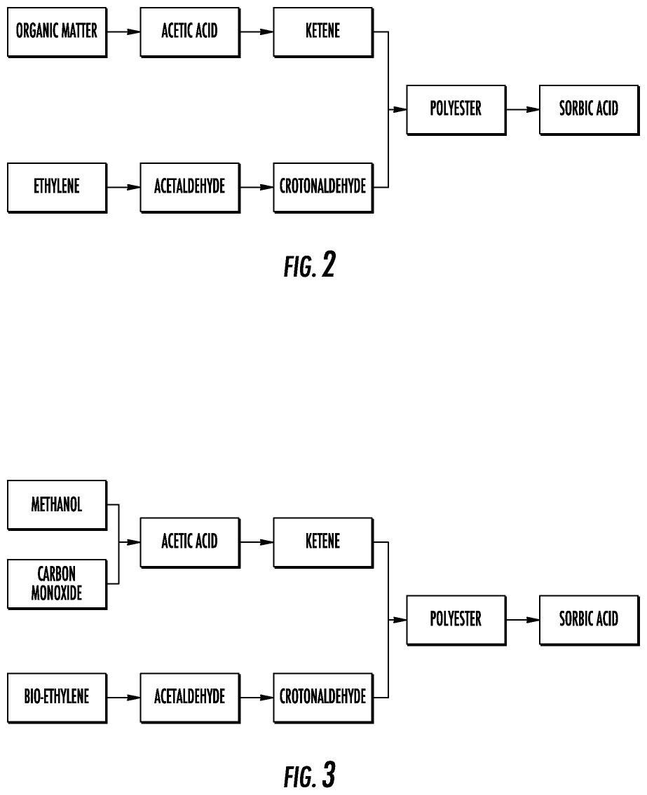

Another embodiment is shown in FIG. 3. The process depicted in FIG. 3 is the same as the process shown in FIG. 1 except that either the ethylene, acetaldehyde, or crotonaldehyde is derived from renewable materials rather than fossil sources and the methanol may be derived from fossil sources rather than renewable materials. However, the methanol can also be renewably sourced as described with reference to FIG. 1.

In some embodiments, bioethylene can be produced by the dehydrogenation of bioethanol, which is produced by fermentation of renewable organic matter, in the presence of a catalyst. However, the bioethylene can be produced in any manner known in the art.

In one embodiment, bioethanol is converted to biothylene via dehydrogenation by contacting ethanol with a dehydration catalyst in either the liquid or gas phase. The catalyst can be an alumina catalyst, such as an alumina pre-treated with an organic silylating agent at elevated temperature or a high-purity activated alumina. Such alumina catalysts can optionally be used in the presence of a magnesium, calcium, zinc, aluminum, or zirconium phosphate. Suitable catalysts can also contain a combination of silica and alumina. For example, Si/Al zeolites can be used, such as ZSM-type zeolites. ZSM-type zeolites can optionally be modified with zirconium and/or phosphorous. In one embodiment, a ZSM-type zeolite catalyst is treated with a hydrogen halide, an organic halide capable of elimination of hydrogen halide, a mineral acid, or a sulfonic acid, such as triflic acid. In another embodiment, a zeolite contains channels or pores formed by cycles or rings of oxygen atoms having 8 and/or 10 elements or members. Other suitable silicate catalysts include MFI-type or MEL-type crystalline silicate catalysts; crystalline silicates having a high Si/Al ratio, and dealuminated crystalline silicates.

The reaction can be carried out in any suitable reactor, such as a fixed bed or fluidized bed reactor. The temperatures and pressures used can vary within wide ranges and can be optimized based on the ethanol concentration of the bioethanol, the type of catalyst used, and the type of reactor used. Inert gasses and solvents can be added to facilitate the reaction.

Exemplary process for dehydrogenating ethanol to ethylene are described in U.S. Pat. Nos. 4,207,424; 4,302,357; 4,232,179; 4,727,214; 4,847,223; 4,873,392; 4,670,620; and 9,061,954; European Patent Nos. 22,640 and 1396481; Japanese Patent Publication No. 2007-290991; and Process Economics Reviews PEP' 79-3 (SRI international) of December 1979; which are entirely incorporated herein by reference.

Bioethylene can also be produced by steam cracking bionaphtha. For example, bionaphtha can be produced from natural triglycerides or fatty acids, such as those contained in cooking oils, food waste, and the like. The crude fats & oils can be refined, either physically or chemically, to remove all non-triglyceride and non-fatty acid components. The refined oils can then be fractionated in both liquid and solid fractions. The solid fraction generally consists of substantially saturated acyl-moieties which can be converted to produce bio-naphtha. For example, the solid fraction can be directly hydrodeoxygenated or can also be hydrolyzed to give fatty acids, potentially mixed with those produced during refining. Then fatty acids can be hydrodeoxygenated or decarboxylated to bio-naphtha. Bionaphtha can then be steam cracked to produce ethylene, as known in the art, such as by mixing the bionaphtha with steam in a ratio of 0.2 to 1.0 kg steam per kg bionaphtha and heating to a temperature from about 750 to about 950° C. at a residence time from about 0.05 to about 0.5 seconds.

Exemplary processes for producing ethylene from bionaphtha are described in U.S. Pat. Nos. 8,624,071 and 8,889,933, which are entirely incorporated herein by reference.

Acetaldehyde can be produced from renewable sources by any method known in the art, such as by converting ethanol to acetaldehyde via oxidative or nonoxidative dehydrogenation. One method is to pass ethanol and air over a silver catalyst at elevated temperatures, e.g., about 400° C. or more. Other suitable catalysts include copper, brass, metal oxides such as CuO, SiO2, ZrO2, Al2O3, MgO, ZnO, and combinations thereof, such as ZnO—CuO—SiO2. Exemplary processes for converting ethanol to acetaldehyde are described in, U.S. Pat. Nos. 1,581,641; 2,682,560; 2,086,702; and 2,384,066; and Kustov, et al. Ethanol to Acetaldehyde Conversion under Thermal and Microwave Heating of ZnO—CuO—SiO2 Modified with WC Nanoparticles. Molecules 2021, 26, 1955; which are entirely incorporated herein by reference.

Biocrotonaldehyde can also be used directly. For example, biocrotonaldehyde is produced commercially by Godavari Biorefineries Ltd.

As such, while FIG. 3 shows a conversion from bioethylene to acetaldehyde and then from acetaldehyde to crotonaldehyde, it should be understood that bioacetaldehyde or biocrotonaldehyde can also be used directly, therefore eliminating the need for converting the bioethylene to acetaldehyde and possibly the need for converting the acetaldehyde to crotonaldehyde.

In the embodiment shown in FIG. 3, the methanol can optionally be derived from fossil sources rather than renewable sources, as in FIG. 1. However, other than the source of the ethylene, acetaldehyde, or crotonaldehyde, and optionally the source of the methanol, the process proceeds in the same way as described above with respect to FIG. 1. In the process of FIG. 3, by using bioethylene, bioacetaldehyde, or biocrotonaldehyde, the renewable carbon content of the resulting sorbic acid is at least 66.7%, as determined by a mass balance using carbon counting or by physical analysis carried out according to ASTM D6866-21 (technically equivalent to ISO 16620-2).

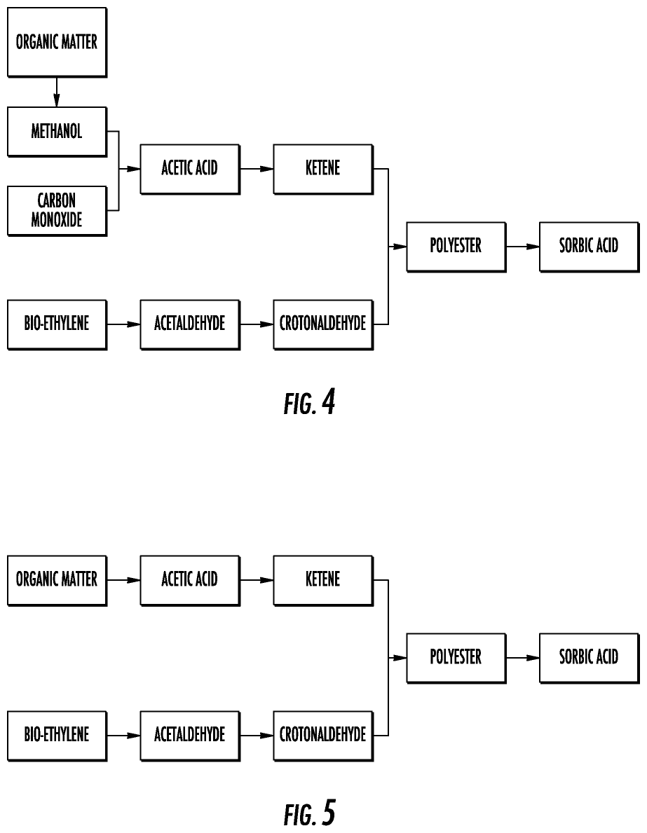

Another embodiment is shown in FIG. 4. As shown, this embodiment combines the use of renewable methanol and the use of bioethylene, bioacetaldehyde, or biocrotonaldehyde. Therefore, the method is a combination of the method illustrated in FIG. 1 with the method illustrated in FIG. 3. As such, the process illustrated by FIG. 4 proceeds in the same manner as described with respect to FIG. 1 except that either the ethylene, acetaldehyde, or crotonaldehyde is derived from renewable sources rather than petrochemical or fossil sources. By combining the renewably sourced methanol with the renewably sourced ethylene, acetaldehyde, or crotonaldehyde, the renewable carbon content in the resulting sorbic acid is at least 83.3%, as determined by a mass balance using carbon counting or by physical analysis carried out according to ASTM D6866-21 (technically equivalent to ISO 16620-2).

Another embodiment is shown in FIG. 5. As shown, this method combines a renewably sourced acetic acid with a renewably sourced ethylene, acetaldehyde, or crotonaldehyde. As such, the embodiment shown in FIG. 5 is a combination of the method shown in FIG. 2 and the method shown in FIG. 3. Other than the sources of the acetic acid and the ethylene, acetaldehyde, or crotonaldehyde, the method shown in FIG. 5 proceeds as described above with reference to FIG. 1. As all the starting materials incorporated into the resulting sorbic acid are produced from renewable sources, the renewable carbon content of the sorbic acid is 100%, as determined by a mass balance using carbon counting or by physical analysis carried out according to ASTM D6866-21 (technically equivalent to ISO 16620-2).

As an additional way to make the sorbic acid production process “greener”, when carbon monoxide is used, such as in the processes shown in FIGS. 1, 3, and 4, the carbon monoxide can be taken from a waste stream or byproduct of another process. Additionally, when non-renewably sourced ethylene is used, such as in the processes shown in FIGS. 1 and 2, it can similarly be taken from a waste stream or byproduct of another process.

The sorbic acid can optionally be neutralized to form a salt, such as an alkali metal salt. For example, commonly used forms are potassium sorbate, sodium sorbate, and potassium sorbate. Such sorbate salts can be formed in any manner known in the art, such as by reacting sorbic acid with alkali metal hydroxides or carbonates in an aqueous medium and crystallizing them. Sorbate salts can be used in any of the same ways as sorbic acid and are sometimes preferred based on the application. For example, when preserving liquid products, potassium sorbate is often preferred due to its high solubility in water.

The sorbic acids or salts thereof obtained as a result of any of the processes described herein can be used in many different applications. For example, they can be used in foods, animal feeds, pharmaceutical drugs, cosmetics, household cleaning products, leather care products, coatings, inks, adhesives, silicone emulsions, surfactants, enzyme preparations, and other water-based products requiring preservation. They can also be used in industrial chemical reactions, for example, as crosslinking agents in the production of rubbers.

The food products can include wines, cheeses, yogurts, apple ciders, fruits, soft drinks, fruit drinks, baked goods, produces, meats, shellfish, prepared salads, and canned goods such as pickles, prunes, cherries, and figs. Sorbic acid and its salts are known as effective preservatives that protect against the growth of bacteria and fungi on such food products.

When used as a food preservative, sorbic acids and sorbates are generally used at concentrations from about 0.025 wt. % to about 0.30 wt. %.

In personal care products, sorbic acids or salts thereof are used as preservatives in skincare, haircare, cosmetics, and body care formulations to extend their shelf lives. For example, they are commonly used in eye and facial makeup products, shampoos, conditioners, moisturizers, sunscreens, and body washes. When used as a preservative in personal care products, sorbic acids and sorbates are generally used at concentrations from about 0.05 wt. % to about 0.30 wt. %.

In pharmaceutical products, sorbic acid and/or salts thereof are used as a preservative in creams, lotions, liquid preparations, nasal drops, nasal sprays, tablets, and the like.

In household cleaning products sorbic acid and/or salts thereof are used as a preservative in washing and cleaning liquids such as hand soaps, dishwashing liquids, fabric detergents, fabric softeners, hard-surface cleaners, and the like. Typical concentrations are from about 0.1% to about 0.8% w/w.

The sorbic acids or salts thereof can be applied to the product intended to be preserved in various ways. For example, they can be applied to the product in a powder form, sprayed onto the surface of the product, incorporated directly, applied to the packaging material of the product, or the product can be dipped into a solution containing the sorbate.

The sorbic acid can also be used to aid in the crosslinking of rubbers, such as EPDM. Such a process is described in U.S. Pat. No. 7,959,530, which is entirely incorporated herein by reference.

As a result of the described processes, food, feed, pharmaceutical, personal care, household cleaning, and leather care products, coatings, inks, adhesives, silicone emulsions, surfactants, enzyme preparations, and other water-based products requiring preservation can contain a preservative including sorbic acid or a salt thereof which contains renewable carbon content. For example, as described above, the renewable carbon content can be greater than about 15%, in some embodiments greater than about 30% in some embodiments greater than about 60%, in some embodiments greater than about 80%, and in some embodiments, 100%, as determined by a mass balance using carbon counting or by physical analysis carried out according to ASTM D6866-21 (technically equivalent to ISO 16620-2).

What is claimed is:

1. A process for making sorbic acid from renewable materials, the process comprising:
converting acetic acid to ketene;
reacting the ketene with crotonaldehyde to produce a polyester; and
converting the polyester to sorbic acid;
wherein a) the acetic acid is produced by reacting methanol derived from renewable organic material with carbon monoxide, b) the acetic acid is a biobased acetic acid, c) the crotonaldehyde is a biobased crotonaldehyde, d) the crotonaldehyde is produced by converting a biobased acetaldehyde to crotonaldehyde, e) the crotonaldehyde is produced by converting acetaldehyde to crotonaldehyde and the acetaldehyde is produced from bioethylene, or any combination of a), b), c), d) and e).

2. The process according to claim 1, wherein converting the acetic acid to ketene comprises pyrolyzing the acetic acid in the presence of a phosphate catalyst.

3. The process according to claim 1, wherein the acetaldehyde or biobased acetaldehyde is converted to crotonaldehyde by aldol condensation of the acetaldehyde using a basic catalyst.

4. The process according to claim 1, wherein reacting the ketene with the crotonaldehyde comprises contacting the ketene and crotonaldehyde with a fatty acid salt of a divalent and/or trivalent metal of subgroup I to VIII of the Periodic Table in the presence of an inert solvent.

5. The process according to claim 1, wherein converting the polyester to sorbic acid comprises carrying out thermal catalytic cleavage of the polyester in the presence of an inert solvent and an amine catalyst.

6. The process according to claim 1, further comprising converting the sorbic acid to potassium sorbate by neutralizing the sorbic acid with potassium hydroxide.

7. The process according to claim 1, wherein the acetic acid is produced by reacting methanol derived from organic waste with carbon monoxide.

8. The process according to claim 1, wherein the acetic acid is a biobased acetic acid formed by oxidizing a biobased ethanol or 2,3-butanediol.

9. The process of claim 1, wherein the acetic acid is a biobased acetic acid recovered from a wood extract.

10. The process according to claim 1, wherein the acetaldehyde is produced from bioethylene by catalytic dehydrogenation of bioethanol.

11. The process according to claim 1, wherein the acetic acid is produced by reacting methanol derived from organic waste with carbon monoxide and the acetaldehyde is produced from bioethylene.

12. The process according to claim 1, wherein the acetic acid is produced by reacting methanol derived from organic waste with carbon monoxide, b) the acetic acid is a biobased acetic acid, and c) the acetaldehyde is produced from bioethylene.

13. The process according to claim 1, wherein about 15% or more of the carbon content in the sorbic acid is biobased, as determined by a mass balance using carbon counting or by physical analysis carried out according to ASTM D6866-21.

14. The process according to claim 1, wherein about 30% or more of the carbon content in the sorbic acid is biobased, as determined by a mass balance using carbon counting or by physical analysis carried out according to ASTM D6866-21.

15. The process according to claim 1, wherein about 60% or more of the carbon content in the sorbic acid is biobased, as determined by a mass balance using carbon counting or by physical analysis carried out according to ASTM D6866-21.

16. The process according to claim 1, wherein about 80% or more of the carbon content in the sorbic acid is biobased, as determined by a mass balance using carbon counting or by physical analysis carried out according to ASTM D6866-21.

17. The process according to claim 1, wherein 100% of the carbon content in the sorbic acid is biobased, as determined by a mass balance using carbon counting or by physical analysis carried out according to ASTM D6866-21.

18. A sorbic acid produced by the process according to claim 1.

19. A potassium sorbate produced by the process according to claim 6.

20. A food, feed, pharmaceutical, personal care, or household cleaning product containing the potassium sorbate of claim 18.

21. A process for making sorbic acid from renewable materials, the process comprising:
converting acetic acid to ketene;
reacting the ketene with crotonaldehyde to produce a polyester; and
converting the polyester to sorbic acid;
wherein about 15% or more of the carbon content in the sorbic acid is biobased, as determined by a mass balance using carbon counting or by physical analysis carried out according to ASTM D6866-21.

22. The process according to claim 21, wherein the acetic acid is produced by reacting methanol derived from renewable organic material with carbon monoxide.

23. The process according to claim 21, wherein the acetic acid is a biobased acetic acid.

24. The process according to claim 21, wherein the crotonaldehyde is produced by converting a biobased acetaldehyde to crotonaldehyde.

25. The process according to claim 21, wherein the crotonaldehyde is produced by converting acetaldehyde to crotonaldehyde and the acetaldehyde is produced from bioethylene.

26. The process according to claim 21, wherein the acetic acid is produced by reacting methanol derived from renewable organic material with carbon monoxide and the crotonaldehyde is produced by converting acetaldehyde to crotonaldehyde, wherein the acetaldehyde is a biobased acetaldehyde or wherein the acetaldehyde is produced from bioethylene.

27. The process according to claim 21, wherein the acetic acid is a biobased acetic acid and the crotonaldehyde is produced by converting acetaldehyde to crotonaldehyde, wherein the acetaldehyde is a biobased acetaldehyde or wherein the acetaldehyde is produced from bioethylene.

28. The process according to claim 21, wherein about 30% or more of the carbon content in the sorbic acid is biobased, as determined by a mass balance using carbon counting or by physical analysis carried out according to ASTM D6866-21.

29. The process according to claim 21, wherein about 60% or more of the carbon content in the sorbic acid is biobased, as determined by a mass balance using carbon counting or by physical analysis carried out according to ASTM D6866-21.

30. The process according to claim 21, wherein about 80% or more of the carbon content in the sorbic acid is biobased, as determined by a mass balance using carbon counting or by physical analysis carried out according to ASTM D6866-21.

31. The process according to claim 21, wherein 100% of the carbon content in the sorbic acid is biobased, as determined by a mass balance using carbon counting or by physical analysis carried out according to ASTM D6866-21.

32. A food, feed, pharmaceutical, personal care, or household cleaning product containing a preservative comprising sorbic acid or an alkali metal salt of sorbic acid, wherein about 15% or more of the carbon content in the sorbic acid or the alkali metal salt of sorbic acid is biobased, as determined by a mass balance using carbon counting or by physical analysis carried out according to ASTM D6866-21.

33. The food, feed, pharmaceutical, personal care, or household cleaning product according to claim 32, wherein about 30% or more of the carbon content in the sorbic acid or the alkali metal salt of sorbic acid is biobased, as determined by a mass balance using carbon counting or by physical analysis carried out according to ASTM D6866-21.

34. The food, feed, pharmaceutical, personal care, or household cleaning product according to claim 32, wherein about 60% or more of the carbon content in the sorbic acid or the alkali metal salt of sorbic acid is biobased, as determined by a mass balance using carbon counting or by physical analysis carried out according to ASTM D6866-21.

35. The food, feed, pharmaceutical, personal care, or household cleaning product according to claim 32, wherein about 80% or more of the carbon content in the sorbic acid or the alkali metal salt of sorbic acid is biobased, as determined by a mass balance using carbon counting or by physical analysis carried out according to ASTM D6866-21.

36. The food, feed, pharmaceutical, personal care, or household cleaning product according to claim 32, wherein 100% of the carbon content in the sorbic acid or the alkali metal salt of sorbic acid is biobased, as determined by a mass balance using carbon counting or by physical analysis carried out according to ASTM D6866-21.

37. The food, feed, pharmaceutical, personal care, or household cleaning product according to claim 32, wherein the preservative comprises potassium sorbate.
